(19) United States
(12) Patent Application Publication
(10) Pub. No.: US 2007/0021627 A1
(43) Pub. Date: Jan. 25, 2007

(54) Process for the preparation of 1-[cyano(4-hydroxyphenyl)methyl]cyclohexanol compounds

(76) Inventors: Frieder Mitzel, Zurich (CH); Beat Theodor Weber, Zofingen (CH); Hans-Rudolf Marti, Oftringen (CH); Richard Haldimann, Aarau (CH)

Correspondence Address: HOFFMANN & BARON, LLP, 6900 JERICHO TURNPIKE, SYOSSET, NY, 11791, US

(21) Appl. No.: 11/478,154

(22) Filed: Jun. 29, 2006

(30) Foreign Application Priority Data
Jun. 29, 2005 (CH) 01101/05

(51) Int. Cl. C07C 253/30

(52) U.S. Cl. 558/357

(57) ABSTRACT

Process for the preparation of I-[cyano(phenyl)methyl]cyclohexanol compounds of general formula (I): 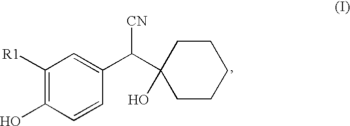 in which
R₁ is hydrogen, (C₁₋₄)alkyl or (C₁₋₄)alkoxy,
   wherein a compound of general formula (II): 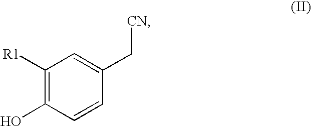
   in which R₁ is as defined above, is reacted with cyclohexanone, the reaction being carried out in the presence of an organic or inorganic base, and this organic or inorganic base being present in the reaction mixture in at least an equimolar amount, based on the amount of the compound of general formula (II).

The present invention relates to a process for the preparation of optionally substituted 1-[cyano(4-hydroxyphenyl)methyl]cyclohexanol compounds, especially the compound 1-[cyano(4-hydroxyphenyl)methyl]cyclohexanol, which is an important intermediate for the preparation of O-demethylvenlafaxin.

In particular, the invention relates to the direct reaction of optionally substituted 4-hydroxyphenylacetonitrile with cyclohexanone. It has so far proved impossible to carry out the direct reaction of 4-hydroxyphenylacetonitrile with cyclohexanone in the presence of a base to give 1-[cyano(4-hydroxyphenyl)methyl]cyclohexanol. 1-[Cyano(4-hydroxyphenyl)methyl]cyclohexanol is therefore prepared using a 4-alkoxyphenylacetonitrile compound, i.e. an acetonitrile compound with a protected hydroxyl group, as the starting compound, the alkoxy group then being converted to the hydroxyl group. Thus there is a need to simplify the preparation of 1-[cyano(4-hydroxyphenyl)methyl]cyclohexanol compounds and to use the optionally substituted 4-hydroxyphenylacetonitrile directly in the reaction. This would make it possible to dispense with the preparation of the 4-alkoxy compound as starting material and with the subsequent conversion of the alkoxy group contained in the compound obtained in the reaction to the hydroxyl group.

The present invention relates to a process for the preparation of 1-[cyano(phenyl)methyl]cyclohexanol compounds of general formula (I):

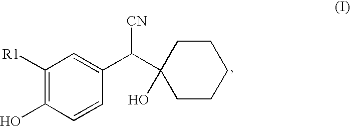

in which

R₁ is hydrogen, (C₁₋₄)alkyl or (C₁₋₄)alkoxy,

characterized in that a compound of general formula (II):

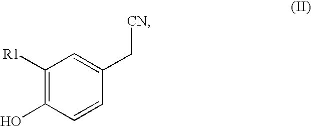

in which R₁ is as defined above, is reacted with cyclohexanone, the reaction being carried out in the presence of an organic or inorganic base, and this organic or inorganic base being present in the reaction mixture in at least an equimolar amount, based on the amount of the compound of general formula (II).

The present invention further relates to the compounds prepared in this way. The present invention further relates to the use of the compound 1-[cyano(4-hydroxyphenyl)methyl]cyclohexanol prepared according to the invention for the preparation of O-demethylvenlafaxin.

The reaction can be performed in the presence of a suitable inert solvent or without the addition of a solvent. Examples of suitable solvents are pentane, hexane, heptane, benzene, toluene, diethyl ether or related solvents. The choice of solvent is familiar to those skilled in the art. Preferably, the reaction is performed without the addition of a solvent.

R₁ is preferably hydrogen or methyl, particularly preferably hydrogen. It is preferable according to the invention to prepare the compound 1-[cyano(4-hydroxyphenyl)methyl]cyclohexanol.

The organic base is preferably selected from the group comprising alkali metal alcoholates, alkaline earth metal alcoholates, aluminium alcoholates and tetrasubstituted ammonium hydroxides, alkali metal and/or alkaline earth metal alcoholates and tetrasubstituted ammonium hydroxides being particularly preferred.

Examples of preferred bases from the group of alkali metal alcoholates are sodium and potassium alcoholates known per se, especially the sodium and potassium alcoholates of methanol, ethanol, n-propanol, sec-propanol, n-butanol, sec-butanol and tert-butanol. The sodium and potassium alcoholates of ethanol and tert-butanol are preferred and sodium tert-butylate and potassium tert-butylate are particularly preferred.

Preferred bases from the group of alkaline earth metal alcoholates are magnesium alcoholates known per se, especially the magnesium alcoholates of methanol, ethanol, n-propanol, sec-propanol, n-butanol, sec-butanol and tert-butanol, the magnesium alcoholates of ethanol and tert-butanol being particularly preferred and magnesium tert-butylate being very particularly preferred.

Preferred bases from the aluminium alcoholates are the aluminium alcoholates of methanol, ethanol, n-propanol, sec-propanol, n-butanol, sec-butanol and tert-butanol, the aluminium alcoholates of ethanol and tert-butanol being particularly preferred and aluminium tert-butylate being very particularly preferred.

Examples of preferred bases from the group of tetrasubstituted ammonium hydroxides are tetra(C₁₋₄)alkylammonium hydroxides such as tetrabutylammonium hydroxide, and tri(C₁₋₄)alkyl(benzyl)ammonium hydroxides such as triethyl(benzyl)ammonium hydroxide. Tetrabutylammonium hydroxide is particularly preferred.

The amount of organic base in the reaction mixture is in the range from at least 1.0 to 2.5 mol, preferably in the range from 1.0 and 2.0 mol and particularly preferably about 1.0 mol per mol of the compound of general formula (II).

The inorganic base is preferably selected from the group comprising alkali metal hydroxides and alkaline earth metal hydroxides and is particularly preferably sodium hydroxide, potassium hydroxide or magnesium hydroxide and very particularly preferably potassium hydroxide, in combination with an alcohol. Preferred alcohols are methanol, ethanol, n-propanol, sec-propanol, n-butanol, sec-butanol and tert-butanol, ethanol and tert-butanol being particularly preferred.

The amount of hydroxide used, preferably sodium hydroxide, potassium hydroxide or magnesium hydroxide and particularly preferably potassium hydroxide, is at least one molar unit (formula unit) of hydroxide per molar unit of the compound of general formula (II), preferably 1.0 molar unit of hydroxide per mol of the compound of general formula (II), and is preferably in the range from 1.0 to 2.5 equivalents of hydroxide per mol of the compound of general formula (II), particularly preferably in the range from 1.0 and 2.0 equivalents and particularly preferably about 1.0 equivalent of hydroxide per mol of the compound of general formula (II). It is not generally critical if a larger excess of hydroxide is present. The alcohol is preferably used in an amount of at least 1 to 5 mol per mol of the compound of general formula (II). It is not generally critical if a larger excess of alcohol is present.

The procedure when using an organic base for the reaction is to mix the two starting materials, i.e. the compound of formula (II) and cyclohexanone, and the base, in any order, at a temperature below 30° C. (<30° C.), and the reaction starts. It is preferable to mix the compound of formula (II) with cyclohexanone and then to add the base. The preferred reaction temperature is in the range from 15° C. to 25° C. The cyclohexanone is preferably used in excess, particularly preferably in an excess of about 1-3 equivalents, based on the compound of formula (II). The reaction time ranges from about 10 minutes to 24 hours, preferably from about 15 minutes to 120 minutes. Then, optionally after the addition of solvent, the product can be isolated and optionally purified further in a manner known per se.

The preferred procedure when using an inorganic base is to choose as the reaction mixture a suitable inert organic solvent which is sufficiently miscible with the alcohol, i.e. which is capable of dissolving the alcohol in an amount of at least 5% by weight, preferably of at least 10% by weight, or is generally miscible with the alcohol. Solid or highly concentrated aqueous alkali metal hydroxide and the starting compounds required for the reaction are added, with cooling, and this reaction mixture is then heated at 40° C.-80° C., preferably at about 50° C.-60° C., preferably for at least 15 minutes. However, the reaction can also be performed without the addition of an organic solvent. Examples of suitable solvents are pentane, hexane, heptane, benzene, toluene, diethyl ether, aprotic solvents or a mixture of these solvents. The choice of solvent is familiar to those skilled in the art.

The Examples which follow illustrate the invention without implying a limitation.

### EXAMPLE 1

8.4 g of potassium tert-butylate are added at room temperature to a solution of 10 g of 4-hydroxybenzyl cyanide in 22.1 g of cyclohexanone. The mixture is stirred for 1.5 hours (h) at room temperature and 100 ml of water and 100 ml of ethyl acetate are then added. The mixture is brought to pH 3-4 with hydrochloric acid and the organic phase is separated off, dried with sodium sulfate and concentrated on a rotary evaporator. Heptane is added to the residue, the mixture is partially concentrated again and a white solid precipitates out. The solid is filtered off, washed with heptane and dried under vacuum to give 11.5 g of 1-[cyano(4-hydroxyphenyl)methyl]cyclohexanol (66% of theory).

### EXAMPLE 2

8.4 g of potassium tert-butylate are added at room temperature to a suspension of 10 g of 4-hydroxybenzyl cyanide and 22.1 g of cyclohexanone in 50 ml of heptane. The mixture is stirred for 18 h at room temperature and 100 ml of water and 100 ml of ethyl acetate are then added. The mixture is brought to pH 3-4 with hydrochloric acid and the organic phase is separated off, dried with sodium sulfate and concentrated to approx. one third of its volume on a rotary evaporator. The white solid obtained is filtered off, washed with heptane and then dried under vacuum to give 11.1 g of 1-[cyano(4-hydroxyphenyl)methyl]cyclohexanol (64% of theory).

### EXAMPLE 3

8.4 g of potassium tert-butylate are added at room temperature to a solution of 10 g of 4-hydroxybenzyl cyanide and 22.5 g of cyclohexanone in 50 g of toluene. The mixture is stirred for 24 h at room temperature, 50 g of water and 20 g of acetic acid are then added and the resulting mixture is refluxed for 30 minutes. The solution is then cooled to room temperature and a white solid crystallizes out. The solid is filtered off, washed with 20 g of toluene and dried under vacuum to give 3.5 g of 1-[cyano(4-hydroxyphenyl)methyl]cyclohexanol (20% of theory).

### EXAMPLE 4

42.4 g of a 25% solution of potassium tert-pentylate in toluene are added dropwise to a solution of 10 g of 4-hydroxybenzyl cyanide and 22.5 g of cyclohexanone in 50 g of toluene, cooled in a water/ice bath. The suspension formed is stirred for 8 h at 0-5° C., 50 g of water and 20 g of acetic acid are then added and the mixture is refluxed for 30 minutes. The solution is then cooled to room temperature and a white solid crystallizes out. The solid is filtered off, washed with 20 g of toluene and . . . under vacuum to give 7 g of 1-[cyano(4-hydroxyphenyl)methyl]-cyclohexanol (20; of theory).

1. Process for the preparation of 1-[cyano(phenyl)methyl]cyclohexanol compounds of general formula (I): 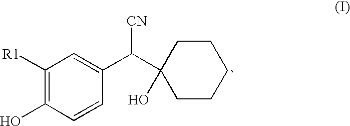
in which
R₁ is hydrogen, (C₁₋₄)alkyl or (C₁₋₄)alkoxy,
characterized in that a compound of general formula (II): 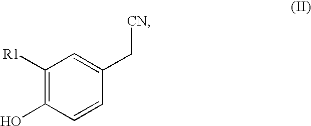
in which R₁ is as defined above, is reacted with cyclohexanone, the reaction being carried out in the presence of an organic or inorganic base, and this organic or inorganic base being present in the reaction mixture in at least an equimolar amount, based on the amount of the compound of general formula (II).

2. Process according to claim 1, characterized in that the reaction is carried out in the presence of an inert solvent or without the addition of a solvent.

3. Process according to claim 2, characterized in that the reaction is carried out in the presence of a solvent selected from the group comprising pentane, hexane, heptane, benzene, toluene and diethyl ether.

4. Process according to claims 1, characterized in that R₁ is hydrogen or methyl, preferably hydrogen.

5. Process according to claims 1, characterized in that the organic base is selected from the group comprising alkali metal alcoholates, alkaline earth metal alcoholates, aluminium alcoholates and tetrasubstituted ammonium hydroxides, preferably alkali metal and/or alkaline earth metal alcoholates and tetrasubstituted ammonium hydroxides.

6. Process according to claim 5, characterized in that the organic base is an alkali metal alcoholate, preferably a sodium or potassium alcoholate, particularly preferably the sodium or potassium alcoholate of methanol, ethanol, n-propanol, sec-propanol, n-butanol, sec-butanol or tert-butanol and very particularly preferably that of ethanol or tert-butanol, especially sodium tert-butylate or potassium tert-butylate.

7. Process according to claim 5, characterized in that the organic base is an alkaline earth metal alcoholate, preferably a magnesium alcoholate, particularly preferably the magnesium alcoholate of methanol, ethanol, n-propanol, sec-propanol, n-butanol, sec-butanol or tert-butanol and very particularly preferably the magnesium alcoholate of ethanol or tert-butanol, especially magnesium tert-butylate.

8. Process according to claim 5, characterized in that the organic base is an aluminium alcoholate, preferably the aluminium alcoholate of methanol, ethanol, n-propanol, sec-propanol, n-butanol, sec-butanol or tert-butanol and particularly preferably the aluminium alcoholate of ethanol or tert-butanol, especially aluminium tert-butylate.

9. Process according to claim 5, characterized in that the organic base is a tetrasubstituted ammonium hydroxide, preferably a tetra(C₁₋₄)alkylammonium hydroxide and particularly preferably tetrabutylammonium hydroxide, or a tri(C₁₋₄)alkyl(benzyl)ammonium hydroxide such as triethyl(benzyl)ammonium hydroxide, tetrabutylammonium hydroxide being preferred.

10. Process according to claims 1, characterized in that the amount of organic base in the reaction mixture is in the range from at least 1.0 to 2.5 mol, preferably in the range from 1.0 and 2.0 mol and particularly preferably about 1.0 mol per mol of the compound of general formula (II).

11. Process according to claim 1, characterized in that the inorganic base is selected from the group comprising alkali metal hydroxides and alkaline earth metal hydroxides and is preferably sodium hydroxide, potassium hydroxide or magnesium hydroxide and particularly preferably potassium hydroxide, and is used in combination with an alcohol, preferably methanol, ethanol, n-propanol, sec-propanol, n-butanol, sec-butanol or tert-butanol and particularly preferably ethanol or tert-butanol.

12. Process according to claim 10, characterized in that the amount of hydroxide used is at least one molar unit of hydroxide per molar unit of the compound of general formula (II) and is preferably in the range from 1.0 to 2.5 equivalents of hydroxide per mol of the compound of general formula (II).

13. Process according to claim 1, characterized in that an organic base is used and the compound of formula (II) and cyclohexanone, and the base, are mixed, in any order, at a temperature below 30° C. (<30° C.), it being preferable to mix the compound of formula (II) with cyclohexanone and then to add the base.

14. Process according to claim 13, characterized in that the reaction temperature is in the range from 15° C. to 25° C., the cyclohexanone is used in excess, preferably in an excess of about 1-3 equivalents, based on the compound of formula (II), and, optionally after the addition of solvent, the product is isolated and optionally purified further in a manner known per se.
